(19) United States
(12) Patent Application Publication
(10) Pub. No.: US 2012/0165220 A1
(43) Pub. Date: Jun. 28, 2012

(54) Uses of CD116 Expression Level

(76) Inventors: Sean P. Colgan, Foxfield, CO (US); Jonathan Goldstein, Pittsford, NY (US)

(21) Appl. No.: 13/393,585

(22) PCT Filed: Aug. 31, 2010
(86) PCT No.: PCT/US10/47289

§ 371 Date: Mar. 1, 2012

Related U.S. Application Data
(60) Provisional application No. 61/239,695, filed on Sep. 3, 2009.

(51) Int. Cl. C40B 30/04; G01N 33/53; G01N 33/566; C12Q 1/68

(52) U.S. Cl. 506/9; 435/6.12; 435/7.1; 435/7.92; 435/7.94; 436/501

(57) ABSTRACT

The present invention provides methods for using granulocyte macrophage colony-stimulating factor receptor (CD116) expression level for various clinical applications including, but not limited to, diagnosing inflammatory bowel disease in a subject.

### CROSS-REFERENCE TO RELATED APPLICATIONS

This application claims the priority benefit of U.S. Provisional Application No. 61/239,695, filed Sep. 3, 2009, which is incorporated herein by reference in its entirety.

### STATEMENT REGARDING FEDERALLY FUNDED RESEARCH

This invention was made with government support under Grant Nos. R37-DK5018 and RO1-HL60569 awarded by the National Institutes of Health. The government has certain rights in the invention.

### FIELD OF THE INVENTION

The present invention relates to methods for using CD116 expression level in a variety of clinical applications including, but not limited to, diagnosing inflammatory bowel disease (IBD) and/or irritable bowel syndrome (IBS) in a subject.

### BACKGROUND OF THE INVENTION

Irritable bowel syndrome (IBS) is a condition that produces some symptoms similar to inflammatory bowel disease (IBD). However, they are not the same condition and they involve very different treatments. IBS affects at least 10-20% of adults in the U.S. and is one of the top ten diagnosed conditions. It is estimated that IBS results in direct and indirect medical costs of over $20 billion annually. Currently the diagnosis of IBS is based on clinical criteria. Other tests such as blood and stool, endoscopy, and x-rays all may be used to help rule out other diseases especially IBD.

IBD is less common than IBS in the U.S. and is estimated to affect approximately 1.5 million Americans. Because IBS and IBD share many similar symptoms, often the two are indistinguishable. Typically, an invasive procedure such as colonoscopy or a CT scan is used to determine the presence or absence of IBD. Such procedures increase the cost of diagnosing IBS or IBD and subject IBS patients to unnecessary risk.

Therefore, there is a need for a less invasive or none invasive and less risky test to diagnose whether a subject is suffering from IBD or IBS.

### SUMMARY OF THE INVENTION

Some aspects of the invention is based on the discovery by the present inventors a correlation between granulocyte macrophage colony-stimulating factor receptor (CD116) expression level and inflammatory bowel disease (IBD) in a subject. Accordingly, one aspect of the invention provides a method for determining the presence of IBD in a subject. Typically, such a method comprises determining the expression level of CD116 in a sample obtained from the subject; and comparing the subject's CD116 expression level with a control CD116 expression level to determine the presence of IBD in the subject.

In some embodiments, the control CD116 expression level comprises a CD116 expression level in a subject without IBD. In such embodiments, a lower expression level of the subject's CD116 compared to the control CD116 expression level is an indication that the subject has IBD. Within these embodiments, any statistically significant difference in the CD116 expression level is an indication that the subject has IBD. Typically, however, the expression level of 50% or less of CD116 in the subject compared to the expression level of CD116 in the control subject is an indication that the subject has IBD.

In other embodiments, the control CD116 expression level comprises a CD116 expression level in a subject with IBD. In these embodiments, a similar expression level of the subject's CD116 compared to the control CD116 expression level is an indication that the subject has IBD. As used herein, a “similar expression level” refers to statistically none significant difference in the CD116 expression level between the subject's sample and the control.

Yet in other embodiments, the sample comprises blood, buccal swab, tissue, urine, or a combination thereof. Still in other embodiments, the sample comprises blood cells. Yet still in other embodiments, the sample comprises granulocytes, monocytes, or a combination thereof.

In some embodiments, the level of CD116 is determined using an antibody for CD116.

Still in other embodiments, the level of CD116 is determined using at least two antibodies capable of identifying CD116, contacting at least a portion of the sample obtained from the subject with the antibodies; and monitoring the extent of reaction between the contacted sample and the antibodies. Within these embodiments, in some instances the contacting and monitoring steps are carried out by extracting the proteins from the sample and conducting an assay to determine the quantity of CD116. Still in other instances of these embodiments, the antigen-antibody binding reaction is detected by a technique comprising enzyme-linked immunosorbent assay (ELISA), radioimmunoassay (RIA), sandwich assay, Western blotting, immunoblotting analysis, an immunohistochemistry method, or a combination thereof. Yet in other instances within these embodiments, the contacting and monitoring steps are carried out by immunohistochemically reacting the sample and the antibodies and then detecting the reactions in the sample.

Yet in other embodiments, methods of the invention can be used to differentiate between ulcerative colitis (UC) and Crohn's disease (CD).

In some embodiments of the invention, the expression level of CD116 is detected using DNA microarray hybridization.

In other embodiments, the expression level of CD116 is detected using polymerase chain reaction (PCR).

Yet other aspects of the invention provide a method for monitoring a patient's response to a treatment for an inflammatory bowel disease (IBD). These aspects of the invention include determining the expression level of granulocyte macrophage colony-stimulating factor receptor (CD116) in a sample taken from the patient. Typically, an increase in the expression level of CD116 of the patient compared to a reference expression level of CD116 is indication that the patient is responding positively to the treatment. Such methods can further include the steps of taking samples during the course of the treatment, and comparing the expression level of CD116 from the previously measured expression level of patient's CD116.

In some embodiments, the reference expression level of CD116 comprises the pretreatment expression level of patient's CD116.

Still other aspects of the invention provide a method for determining a need for an invasive medical procedure to distinguish between inflammatory bowel disease (IBD) and irritable bowel syndrome (IBS) in a subject. Such aspects of the invention include determining the expression level of the subject's granulocyte macrophage colony-stimulating factor receptor (CD 116); and determining a need for an invasive medical procedure by comparing the subject's CD116 expression level with the control CD116 expression level. The control CD116 expression level can be the CD116 expression level of normal (i.e., subjects without IBD) or the CD116 expression level of subjects with IBD.

Yet still other aspects of the invention provide a method for identifying a drug candidate for treating inflammatory bowel disease (IBD). Such aspects of the invention typically include:
(a) administering a test compound to a mammal having an IBD, or to intestinal cells isolated from a mammal with IBD;
(b) obtaining a sample from the mammal or the cell composition;
(c) determining the level or the gene expression level of granulocyte macrophage colony-stimulating factor receptor (CD116) in the sample; and
(d) comparing the level or the gene expression level of CD116 from the sample with either or both of the following:
   (i) the level or the gene expression level of CD116 in the same mammal or the same intestinal cells in the absence of the test compound; or
   (ii) the level or the gene expression level of CD116 in a normal mammal or in normal cells;
      wherein test compounds that increase the level or the gene expression level of CD116 to approach the level or the gene expression level of CD116 seen in the normal mammal or the normal cells are thereby identified as candidates for drugs to treat IBD.

Still other aspects of the invention provide a method for distinguishing between the presence of inflammatory bowel disease (IBD) from other clinical condition in a patient. Such methods include determining the expression level of granulocyte macrophage colony-stimulating factor receptor (CD116) in a sample obtained from the subject; and comparing the subject's CD116 expression level with a control CD116 expression level to distinguishing between the presence of inflammatory bowel disease (IBD) from other clinical condition in the patient.

In some embodiments, the other clinical condition comprises irritable bowel syndrome (IBS), celiac disease, ischemic colitis, infectious colitis, or a combination thereof.

### BRIEF DESCRIPTION OF THE DRAWINGS

FIGS. 1A and 1B show bar graph analysis of mRNA expression of CD116 (GM-CSF receptor) and CD114 (G-CSF receptor), respectively, in healthy and IBD granulocytes. Total RNA was isolated from purified granulocytes derived from healthy control (n=10) or IBD (n=10) patients. RNA was used as a template to analyze CD116 (panel A) and CD114 (panel B) relative to β-actin using real-time PCR.

FIGS. 2A-2C show flow cytometric analysis of CD116 surface expression on healthy and IBD granulocytes and monocytes, respectively. Anti-coagulated whole venous blood was obtained from healthy controls (n=52) and IBD patients (n=52) and used to examine CD116 expression by flow cytometry. FIG. 2A shows an example of the gating strategy used to define granulocytes and monocytes. FIGS. 2B and 2C show expression levels (MFI, mean fluorescence intensity) for granulocytes and monocytes, respectively.

FIGS. 3A-3D show receiver operating characteristic (ROC) curve analysis for CD116 expression on healthy, irritable bowel syndrome (IBS) and IBD leukocytes. Anti-coagulated whole venous blood was obtained from healthy controls (closed circles, n=52) irritable bowel patients (IBS, shown in open squares, n=7) and IBD patients (closed triangles, n=52) and used to examine CD116 expression by flow cytometry. FIGS. 3A and 3B show expression of CD116 for granulocytes and monocytes, respectively. FIGS. 3C and 3D depict ROC curve analysis for granulocytes and monocytes, respectively.

FIGS. 4A-4D show influence of disease activity and current medications on CD116 expression on IBD leukocytes. Anti-coagulated whole venous blood was obtained from IBD patients with indicated disease activity (FIGS. 4A and 4B) or on indication medications (FIGS. 4C and 4D) and examined for surface expression of CD116 on granulocytes (FIGS. 4A and 4C) or monocytes (FIGS. 4B and 4D), respectively.

FIGS. 5A and 5B show analysis of CD116 expression on UC and CD leukocytes. Anti-coagulated whole venous blood was obtained from UC or CD patients and examined for surface expression of CD116 on granulocytes (FIG. 5A) or monocytes (FIG. 5B), respectively.

FIGS. 6A-6C show comparison of CD116 expression and GM-CSF-stimulated STAT3 phosphorylation in healthy and IBD granulocytes. Purified granulocytes from healthy controls (n=10) and IBD patients (n=8) were exposed to rhGM-CSF (180 ng/ml, 15 min) and stained for CD116 (FIG. 6A) or pSTAT3 (FIG. 6B). FIG. 6C shows a plot of CD116 vs pSTAT3 for IBD (closed squares) and healthy control granulocytes (open circles).

### DETAILED DESCRIPTION OF THE INVENTION

Irritable bowel syndrome (IBS) refers to a complex disorder of the lower intestinal tract. It is mainly characterized by a pattern of symptoms that is often worsened by emotional stress. While symptoms of IBS range from mild to severe, most people have mild symptoms. IBS symptoms may be worse in patients with underlying stress or mood disorders such as anxiety and depression. Symptoms may include: abdominal distention; abdominal fullness, gas, bloating; abdominal pain or tenderness that comes and goes, goes away after a bowel movement, and/or occurs after meals; chronic and frequent constipation, usually accompanied by pain; chronic and frequent diarrhea, usually accompanied by pain; emotional distress; depression; loss of appetite; and nausea and vomiting among others.

IBS involve a combination of abdominal pain and alternating constipation and diarrhea. There are many possible causes. For instance, there may be a problem with muscle movement in the intestine or a lower tolerance for stretching and movement of the intestine. Generally, there is no problem in the structure of the intestine. It is not clear why patients develop IBS, but in some instances, it occurs after an intestinal infection. This is called postinfectious IBS. There may also be other triggers. IBS can occur at any age, but often begins in adolescence or early adulthood. It is more common in women. The condition is the most common intestinal complaint that leads to referral to a gastroenterologist.

Most of the time, doctors can diagnose IBS without ordering many tests. Tests usually reveal no problems. Some patients may need an endoscopy, especially if symptoms begin later in life. Younger patients with persistent diarrhea may need this test to look for inflammatory diseases that can cause similar symptoms such as Crohn's disease or ulcerative colitis. Additional tests may be needed if the patient has blood in the stool, loses weight, or exhibits signs of anemia.

Although there is no cure for IBS, there are treatments which attempt to relieve symptoms, including dietary adjustments, medication and psychological interventions. Several conditions may present as IBS including celiac disease, mild infections, parasitic infections like giardiasis, several inflammatory bowel diseases, functional chronic constipation, and chronic functional abdominal pain. In IBS, routine clinical tests yield no abnormalities, though the bowels may be more sensitive to certain stimuli, such as balloon insufflation testing. The exact cause of IBS is unknown. It is generally believed that IBS is a disorder of the interaction between the brain and the gastrointestinal tract, although there may also be abnormalities in the gut flora or the immune system.

IBS does not lead to more serious conditions in most patients. But it is a source of chronic pain, fatigue and other symptoms, and it increases a patient's medical costs, and contributes to work absenteeism. Researchers have reported that the high prevalence of IBS, in conjunction with increased costs produces a disease with a high societal cost. It is also regarded as a chronic illness and can dramatically affect the quality of a sufferer's life.

Currently, there is no specific laboratory or imaging test which can be performed to diagnose IBS. Diagnosis of IBS typically involves excluding conditions which produce IBS-like symptoms, and then following a procedure to categorize the patient's symptoms.

Because there are many causes of diarrhea that give IBS-like symptoms, the American Gastroenterological Association has published a set of guidelines for tests to be performed to rule out other causes for these symptoms. These include gastrointestinal infections, lactose intolerance and celiac disease. Research has suggested that these guidelines are not always followed. Once other causes have been excluded, the diagnosis of IBS is performed using a diagnostic algorithm. Well-known algorithms include the Manning Criteria, the obsolete Rome I and II criteria, the Kruis Criteria, and studies have compared their reliability. The algorithm may include additional tests to guard against misdiagnosis of other diseases as IBS. Such “red flag” symptoms include weight loss, GI bleeding, anemia, or nocturnal symptoms. However, researchers have noted that red flag conditions do not always contribute to accuracy in diagnosis—for instance, as many as 31% of IBS patients have blood in their stool.

Inflammatory bowel disease (IBD) is a group of inflammatory conditions (e.g., chronic mucosal inflammation disorders) of the colon and small intestine. IBD symptoms include abdominal cramps, bloody diarrhea, fever, and weight loss. The major types of IBD are Crohn's disease and ulcerative colitis (UC), which causes inflammation in the digestive tract. The main difference between Crohn's disease and UC is the location and nature of the inflammatory changes. Crohn's disease can affect any part of the gastrointestinal tract, from mouth to anus (skip lesions), although a majority of the cases start in the terminal ileum. Ulcerative colitis, in contrast, is restricted to the colon and the rectum. Microscopically, ulcerative colitis is restricted to the mucosa (epithelial lining of the gut), while Crohn's disease affects the whole bowel wall.

Although the etiology of CD and UC remains unclear, accumulating evidence suggests that dysfunction of the mucosal immune system plays a significant role in the pathogenesis of IBD. Significant evidence, in fact, indicates impaired innate immunity (granulocytes, macrophages and dendritic cells) in IBD, particularly CD. These defects include reduced barrier function, delayed bacterial clearance, hyporesponsive neutrophils and defective macrophage cytokine secretion. Such defects have the potential to allow abnormal microbial invasion and pathological T-cell mediated chronic inflammation. Without being bound by any theory, it is also believed that some instances of IBD result from environmental factors (e.g., bacterial antigens) triggering a dysregulated immune response in genetically predisposed hosts.

Although very different diseases, Crohn's disease and UC both may present with any of the following symptoms: abdominal pain, vomiting, diarrhea, hematochezia (bright red blood in stools), weight loss and various associated complaints or diseases like arthritis, pyoderma gangrenosum, and primary sclerosing cholangitis. More accurate diagnosis is generally by colonoscopy with biopsy of pathological lesions.

Since IBD and IBS share many similar symptoms they are often difficult to distinguish one from the other. Currently, accurate diagnosis of IBD typically involves colonoscopy or CT scan. Many IBS patients are often subjected to these unnecessary procedures in order to eliminate IBD as a possible source of their symptoms. If a simple test is available that could help confirm the clinical suspicion of IBS, such a test would eliminate a need for costly and invasive procedures such as colonoscopy and CT scans for patients suffering from IBS. If such a test suggests a likelihood of IBD, a more invasive procedure such as colonoscopy can be performed to confirm IBD diagnosis.

Some aspects of the present invention provide methods for diagnosing IBD. Such methods can also be used to distinguish whether a patient is suffering from IBS or IBD. Some embodiments of such methods comprise determining the expression level of granulocyte macrophage colony-stimulating factor receptor (GM-CSF-R or CD116) in a sample obtained from the subject. As used herein, the term “expression” refers to (1) detecting transcription and/or translation of CD116 gene, (2) detecting or determining the amount of CD116 present in the sample, or (3) both. To detect expression of a gene refers to the act of actively determining whether a gene is expressed or not. This can include determining whether the gene expression is upregulated as compared to a control, downregulated as compared to a control, or substantially unchanged as compared to a control. Therefore, the step of detecting expression does not require that expression of the gene actually is upregulated or downregulated, but rather, can also include detecting no expression of the gene or detecting that the expression of the gene has not changed or is not different (i.e., detecting no significant expression of the gene or no significant change in expression of the gene as compared to a control).

The GM-CSF receptor (also called CD116) belongs to the family of colony-stimulating hematopoietic growth factor receptors, which includes three known structurally distinct receptors that bind GM-CSF, M-CSF or G-CSF. The M-CSF receptor (CSF1R; also known as CD115 or FMS) is a homodimeric type III receptor tyrosine kinase. CD116 (also called CSF2R) consists of a unique a-chain and a common β-chain (βc) which is shared with the interleukin (IL)-3 and IL-5 receptor. The G-CSF receptor (CSF3R or CD114) is typical of the type I cytokine receptor family. CD116 is most prominently expressed on myeloid lineages and on epithelial cells.

It has been found by the present inventors that a lower expression level of CD116 compared to the level of CD116 in a control subject without IBD is an indication that the subject has IBD. Alternatively, if the expression level of CD116 in a subject is similar to the CD116 expression level of other IBD patients, then it is also an indication that the subject has IBD. Methods of the invention allow one to distinguish whether a patient is suffering from IBD or IBS without a need for an invasive or a costly procedure such as colonoscopy or CT scan. It should be appreciated that levels of other biomarkers such as any portion of protein, mRNA, gene expression, or ligand that can identify or correlate with the level of CD116 can be used in the diagnosis. In addition, in some instances phospho-STAT3 can also be used (alone or in combination with CD116) as a biomarker for determining the presence of IBD in a subject.

Methods of the invention include detecting or determining whether there is any significant difference in the expression level of CD116 in the tested subject compared to the expression level of CD116 in a control. According to the invention, a “baseline” or “control” can include a normal or negative control and/or a disease or positive control, against which a test level of CD116 expression can be compared. Therefore, it can be determined, based on the control or baseline expression level of CD116, whether a sample to be evaluated for IBD has a measurable difference or substantially no difference in the CD116 expression level, as compared to the baseline level. In one aspect, the baseline control is an indicative of the expression level of CD116 as expected in a normal (e.g., healthy, negative control, non-IBD) patient. Therefore, the term “negative control” used in reference to a baseline expression level of CD116 typically refers to a baseline expression level of CD116 from a population of individuals which is believed to be normal (i.e., not having IBD).

In some embodiments of the invention, a patient's test sample is compared to a baseline CD116 expression level that has previously been established from a patient or population of patients with IBD. Such a baseline expression level, also referred to herein as a “positive control”, refers to an expression level of CD116 established from one or often a population of individuals who had been positively diagnosed with IBD.

In some embodiments, the control or baseline expression levels of CD116 are obtained from “matched individuals”. The phrase “matched individuals” refers to a matching of the control individuals on the basis of one or more characteristics, such as gender, age, race, or any relevant biological or sociological factor that may affect the baseline of the control individuals and the patient (e.g., preexisting conditions, consumption of particular substances, levels of other biological or physiological factors). The number of matched individuals from whom control samples must be obtained to establish a suitable control level (e.g., a population) can be determined by those of skill in the art, but should be statistically appropriate to establish a suitable baseline for comparison with the patient to be evaluated (i.e., the test patient). The values obtained from the control samples are statistically processed using any suitable method of statistical analysis to establish a suitable baseline level using methods standard in the art for establishing such values. It will be appreciated by those of skill in the art that a baseline need not be established for each assay as the assay is performed but rather, a baseline can be established by referring to a form of stored information regarding a previously determined control expression level of CD116. Such a form of stored information can include, for example, but is not limited to, a reference chart, listing or electronic file of population or individual data regarding “normal” (negative control) or IBD-positive CD116 expression level; a medical chart for the patient recording data from previous evaluations; or any other source of data regarding control CD116 expression level that is useful for the patient to be diagnosed or evaluated.

Expression of the transcripts and/or proteins encoded by the CD116 genes can be measured by any of a variety of known methods in the art. In general, the nucleic acid sequence of a nucleic acid molecule (e.g., DNA or RNA) in a patient sample can be detected by any suitable method or technique of measuring or detecting gene sequence or expression. Such methods include, but are not limited to, polymerase chain reaction (PCR), reverse transcriptase-PCR (RT-PCR), in situ PCR, quantitative PCR (q-PCR), in situ hybridization, Southern blot, Northern blot, sequence analysis, microarray analysis, detection of a reporter gene, or other DNA/RNA hybridization platforms. For RNA expression, typical methods include, but are not limited to: extraction of cellular mRNA and Northern blotting using labeled probes that hybridize to transcripts encoding all or part of CD116 gene; amplification of mRNA expressed from CD116 gene using gene-specific primers, polymerase chain reaction (PCR), quantitative PCR (q-PCR), and reverse transcriptase-polymerase chain reaction (RT-PCR), followed by quantitative detection of the product by any of a variety of means; extraction of total RNA from the cells, which is then labeled and used to probe cDNAs or oligonucleotides encoding all or part of the genes of this invention, arrayed on any of a variety of surfaces; in situ hybridization; and detection of a reporter gene. The term “quantifying” or “quantitating” when used in the context of quantifying transcription levels of CD116 gene can refer to absolute or to relative quantification. Absolute quantification may be accomplished by inclusion of known concentration(s) of one or more target nucleic acids and referencing the hybridization intensity of unknowns with the known target nucleic acids (e.g. through generation of a standard curve). Alternatively, relative quantification can be accomplished by comparison of hybridization signals to quantify the changes in hybridization intensity and, by implication, transcription level.

Methods to measure protein expression levels of CD116 gene include, but are not limited to, Western blot, immunoblot, enzyme-linked immunosorbant assay (ELISA), radioimmunoas say (RIA), immunoprecipitation, surface plasmon resonance, chemiluminescence, fluorescent polarization, phosphorescence, immunohistochemical analysis, matrix-assisted laser desorption/ionization time-of-flight (MALDI-TOF) mass spectrometry, microcytometry, microarray, microscopy, fluorescence activated cell sorting (FACS), flow cytometry, and assays based on a property of CD116 including but not limited to ligand binding, or interaction with other protein partners.

Nucleic acid arrays can also be used for detecting the expression of CD116 gene. The production and application of arrays in gene expression monitoring have been disclosed previously in, for example, PCT Publication Nos. WO 97/10365, WO 92/10588, WO 95/35505, U.S. Pat. Nos. 6,040,138 and 5,445,934, Hacia et al. (1996) Nature Genetics 14:441-447, Lockhart et al. (1996) Nature Biotechnol. 14:1675-1680, and De Risi et al. (1996) Nature Genetics 14:457-460, all of which are incorporated herein by reference in their entirety. In general, in an array, an oligonucleotide, a cDNA, or genomic DNA, that is a portion of CD116 gene, occupies a known location on a substrate. A nucleic acid target sample is hybridized with an array of such oligonucleotides and then the amount of target nucleic acids hybridized to each probe in the array is quantified. One preferred quantifying method is to use confocal microscope and fluorescent labels. The Affymetrix GeneChip™ Array system (Affymetrix, Santa Clara, Calif.) and the Atlas™ Human cDNA Expression Array system are particularly suitable for quantifying the hybridization; however, it will be apparent to those of skill in the art that any similar systems or other effectively equivalent detection methods can also be used. One can use the knowledge of CD116 gene to design arrays of polynucleotides, cDNAs or genomic DNAs for screening methods described herein. Such novel pluralities of polynucleotides are contemplated to be a part of the invention.

In general, typical clinical samples include, but are not limited to, blood or blood cells such as white blood cells (e.g., granulocytes and monocytes), buccal swabs, tissues, urine, saliva, etc.

The expression level of CD116 can also be determined by conjugation or ligand-binding interaction using a CD116 ligand and/or CD116 antibody that is detectably marked. Detectable markers suitable for use in the invention include any composition detectable by spectroscopic, photochemical, biochemical, immunochemical, electrical, optical or chemical means. Useful labels in the present invention include biotin for staining with labeled streptavidin conjugate, magnetic beads (e.g., Dynabeads.™.), fluorescent dyes (e.g., fluorescein, texas red, rhodamine, green fluorescent protein, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and colorimetric labels such as colloidal gold or colored glass or plastic (e.g., polystyrene, polypropylene, latex, etc.) beads.

Another embodiment of the invention relates to a plurality of antibodies, or antigen binding fragments thereof, for the detection of CD116 in samples of the subject. The plurality of antibodies, or antigen binding fragments thereof, consists of antibodies, or antigen binding fragments thereof, that selectively bind to CD 116. In addition, the plurality of antibodies, or antigen binding fragments thereof, comprises antibodies, or antigen binding fragments thereof, that selectively bind to CD116 or portions thereof.

The phrase “selectively binds to” refers to the ability of an antibody, antigen binding fragment or binding partner (antigen binding peptide) to preferentially bind to CD116. Often the phrase “selectively binds” refers to the specific binding of CD116 (e.g., an antibody, fragment thereof, or binding partner to an antigen), wherein the level of binding, as measured by any standard assay (e.g., an immunoassay), is statistically significantly higher than the background control for the assay. For example, when performing an immunoassay, controls typically include a reaction well/tube that contain antibody or antigen binding fragment alone (i.e., in the absence of antigen), wherein an amount of reactivity (e.g., non-specific binding to the well) by the antibody or antigen binding fragment thereof in the absence of the antigen is considered to be background. Binding can be measured using a variety of methods standard in the art including enzyme immunoassays (e.g., ELISA), immunoblot assays, etc.).

Limited digestion of an immunoglobulin with a protease may produce two fragments. An antigen binding fragment is referred to as an Fab, an Fab′, or an F(ab′)₂ fragment. A fragment lacking the ability to bind to antigen is referred to as an Fc fragment. An Fab fragment comprises one arm of an immunoglobulin molecule containing a L chain (V_{L}+C_{L} domains) paired with the V_{H} region and a portion of the C_{H} region (CH1 domain). An Fab′ fragment corresponds to an Fab fragment with part of the hinge region attached to the CH1 domain. An F(ab′)₂ fragment corresponds to two Fab' fragments that are normally covalently linked to each other through a di-sulfide bond, typically in the hinge regions.

Isolated antibodies of the invention can include serum containing such antibodies, or antibodies that have been purified to varying degrees. Whole antibodies of the invention can be polyclonal or monoclonal. Alternatively, functional equivalents of whole antibodies, such as antigen binding fragments in which one or more antibody domains are truncated or absent (e.g., Fv, Fab, Fab′, or F(ab)₂ fragments), as well as genetically-engineered antibodies or antigen binding fragments thereof, including single chain antibodies or antibodies that can bind to more than one epitope (e.g., bi-specific antibodies), or antibodies that can bind to one or more different antigens (e.g., bi- or multi-specific antibodies), can also be employed in the invention.

Generally, in the production of an antibody, a suitable experimental animal, such as, for example, but not limited to, a rabbit, a sheep, a hamster, a guinea pig, a mouse, a rat, or a chicken, is exposed to an antigen against which an antibody is desired. Typically, an animal is immunized with an effective amount of antigen that is injected into the animal. An effective amount of antigen refers to an amount needed to induce antibody production by the animal. The animal's immune system is then allowed to respond over a pre-determined period of time. The immunization process can be repeated until the immune system is found to be producing antibodies to the antigen. In order to obtain polyclonal antibodies specific for the antigen, serum is collected from the animal that contains the desired antibodies (or in the case of a chicken, antibody can be collected from the eggs). Such serum is useful as a reagent. Polyclonal antibodies can be further purified from the serum (or eggs) by, for example, treating the serum with ammonium sulfate.

Monoclonal antibodies can be produced according to the methodology of Kohler and Milstein (Nature, 1975, 256, 495-497). For example, B lymphocytes are recovered from the spleen (or any suitable tissue) of an immunized animal and then fused with myeloma cells to obtain a population of hybridoma cells capable of continual growth in suitable culture medium. Hybridomas producing the desired antibody are selected by testing the ability of the antibody produced by the hybridoma to bind to the desired antigen.

Methods of the invention can include a step of comparing the results of detecting or determining the CD116 expression level in the subject with the CD116 expression level in a control (baseline, normal control or patient with IBD) in order to determine whether there is any difference in the CD 116 expression level in the subject as compared to the control. As discussed herein, the CD116 expression level can be compared to a “normal” or “negative” control (i.e., a subject that does not have IBD) or a “positive” control (i.e., a subject that has IBD). Therefore, one can determine whether the CD116 expression level from the test subject is statistically substantially similar to the CD116 expression level of subjects with IBD or whether the CD116 expression level in the test subject is statistically more similar to the negative or normal, non-IBD control.

In general, the CD116 expression level is substantially similar to a given CD116 expression level established for a group (e.g., IBD group, or normal or “negative” control group) if the CD116 expression level determined or detected is similar enough to the expected result so as to be statistically significant (e.g., with at least a 95% confidence level, or p<0.05, typically with a confidence level of p<0.01, and often with a confidence level of p<0.005, and more often with a confidence level of p<0.001). Software programs are available in the art that are capable of analyzing whether the difference between the CD 116 expression level from the test subject and a control is significant or not significant. In addition, statistical analysis methods are well known in the art.

The CD116 expression level in a patient can be used by the patient or physician for decision-making regarding the usefulness of therapies for IBD in general. The CD116 expression level can be used to estimate how the disease is likely to respond and progress in any individual patient. The CD116 expression level can also be used to determine patient's response to a particular treatment.

Additional objects, advantages, and novel features of this invention will become apparent to those skilled in the art upon examination of the following examples thereof, which are not intended to be limiting. In the Examples, procedures that are constructively reduced to practice are described in the present tense, and procedures that have been carried out in the laboratory are set forth in the past tense.

### EXAMPLES

### Material and Methods

The diagnosis of IBD was made by the physician caring for the patient based on endoscopic, radiographic and histological criteria. CD and UC patients were diagnosed using established criteria, and CD phenotype was per the Montreal criteria, see for example, Satsangi et al., Gut 2006, 55, 749-53, (see Table 1 below for patient characteristics). IBS patients were diagnosed by ROME III criteria (Drossman et al., Gastroenterology, 2006, 130, 1377-90). Medications were recorded at time of blood draw and the attending physician recorded a clinical estimation of disease activity, which included disease remission, mild disease, moderate disease or severe disease.

TABLE 1
IBD Patient Characteristics

| | Crohn's Disease | Ulcerative Colitis |
|---|---|---|
| No. of Patients | 33 | 19 |
| % Male | 61 | 53 |
| Median Age (yrs) | 31 | 51 |
| Disease Severity | | |
| % Severe | 12 | 5 |
| % Moderate | 36 | 16 |
| % Mild | 40 | 48 |
| % Remission | 12 | 31 |
| Medications | | |
| % None | 15 | 27 |
| % 5-ASA | 27 | 52 |
| % 5-ASA/Steroids | 22 | 15 |
| % Immunosuppressed | 36 | 6 |

### Granulocyte Isolation, RNA Isolation and Transcriptional Analysis

Whole venous blood granulocytes were isolated as described by Weissmuller et al., in J. Clin. Invest., 2008, 118, 3682-92. RNA was isolated using Trizol (Invitrogen, Carlsbad, Calif.) and cDNA synthesized as also described by Weissmuller et al. Potential contaminating genomic DNA was digested using Turbo DNA-free (Ambion, Austin, Tex., USA). Changes in gene expression in granulocytes isolated from patient whole blood were compared to that of granulocytes isolated from healthy controls. Semi-quantitative and real-time PCR were performed using increasing numbers of cycles of 95° C. for 45 sec, 59° C. for 35 sec and 72° C. for 45 sec and a final extension time of 7 min. The following primers were used to quantify expression in isolated granulocytes: CSF2R (CD116): forward 5′-GCAGACGTCCGCATCTTGA-3′ and reverse 5′-CCGTCGTCAGAACCAAATTCA-3′; CSF3R (CD114): forward 5′-AGCCCCAAGTCCTATGAGAAC-3′ and reverse 5′-GCAGGAGGGGGAAGTTGAG-3′; β-actin: forward 5′-GCACTCTTCCAGCCTTCCTTCC-3′ and reverse 5′-CAGGTCTTTGCGGATGTCCACG-3′. Transcript levels and fold change in mRNA were determined as described by Eltzschig et al., J. Ex. Med., 2003, 198, 783-796. Intron-spanning primer pairs were designed using Primer3 software (http://frodo.wi.mit.edu/). Primer properties and secondary structures including hairpins, self-dimers, and cross-dimers were evaluated in a second step using Netprimer software (http://www.premierbiosoft.com/netprimer). Samples were controlled for β-actin.

### GM-CSF Receptor (CD116) Staining

Whole blood (100 μL) was incubated with phycoerythrin-labeled mouse anti-human CD116 (2.5 μL) (BD Pharmigen, San Jose, Calif.) in the dark for 30 minutes at 4° C. The red blood cells (RBC) were then lysed using BD pharm lyse™ (2 mL) (BD Pharmigen, San Jose, Calif.) for 15 minutes in the dark at room temperature. The cells were then washed with FACS wash (phosphate-buffered saline (PBS) and 1% bovine serum albumin) and fixed in 2% paraformaldehyde. CD116 expression on granulocytes and monocytes was evaluated using flow cytometry (FACSCantoII and Diva software, BD Biosciences, San Jose, Calif.). Granulocytes and monocytes were gated according to forward- and side-scatter properties. CD116 expression was expressed as mean fluorescent intensity (MFI).

### CD116 (GMCSF Receptor) ELISA

In the following procedure for CD116 ELISA analysis, capture antibody (GM-CSF Receptor alpha Antibody) was obtained from Novus Biologics Inc. and was used at 0.4 mg/m˜30 μl/well (12 μg/well). The detection antibody (GM-CSF Receptor alpha Antibody) was also obtained from Novus Biologics Inc. and labeled with MSD SULFO-TAG NHS-Ester. A stock solution of the detection antibody was made at 0.2 mg/ml and kept in dark until use. The detection antibody was used at 0.002 mg/ml-30 μ/well (0.06 μg/well). The standard sample (GM-CSF Receptor alpha Recombinant Protein) was also obtained from Novus Biologics Inc.

Briefly, a high binding plate (MesoScale Diagnostics, Gaithersburg, Md.) was coated with anti-GMCSF (30 μl per well, 0.4 mg/ml) at 4° C.. The plate was blocked with 3% Blocker A/PBS (30 μl/well) for 2 hr at RT. The plate was washed 3 times with 150 μl/well of 0.05% PBST. The samples and standards were prepared and diluted with MSD buffer (30 μl per well, 1.6 -3.2 mg/ml samples) Standard curve (3 ng/ml, 3-fold dilution). The standards and samples were added to the plate (30 μl per well) and incubate at 4° C.. The plate was then washed 3 times with 150 μl/well of 0.05% PBST, and the capture antibody (diluted with 1% Blocker A/PBS-0.002 mg/ml) was added (30 μl per well) and incubate for 90 min at RT on a shaker. Read buffer was prepared and 4 part read buffer was diluted with 1 part deionized water. The plate was washed 3 times with 150 μl/well of 0.05% PBST. Immediately before reading, 150 μl/well of diluted read buffer was added while avoiding air bubbles.

Granulocyte phospho-STAT3 Response to GM-CSF

Whole blood (100 μL) was stimulated with PBS or recombinant human GM-CSF (Invitrogen, Carlsbad, Calif.), 180 ng/mL, at 15 minutes. The reaction was fixed and the RBC lysed using BD™ Phosflow Lyse/Fix buffer (2 mL) (BD Biosciences, San Jose, Calif.) for 15 minutes at 37° C.. The cells were permeabilized with BD™ Phosflow Perm Bufffer III (1 ml, BD Biosciences, San Jose, Calif.) for 30 minutes on ice. The tubes were washed and stained with phosphorylated signal transducers and activators of transcription 3 (pSTAT3-pY705) (BD Pharmigen, San Jose, Calif.) for 30 min at room temperature. The granulocytes containing pSTAT3 were determined using flow cytometry.

### Statistical Analysis

All expression patterns were compared using analysis of variance with post-hoc Student's t Test for continuous variables. A p value of <0.05 was considered statistically significant. All data are presented as the mean ±standard error of the mean. Statistical analyses were performed using GraphPad Prism software (LaJolla, Calif.).

### Results

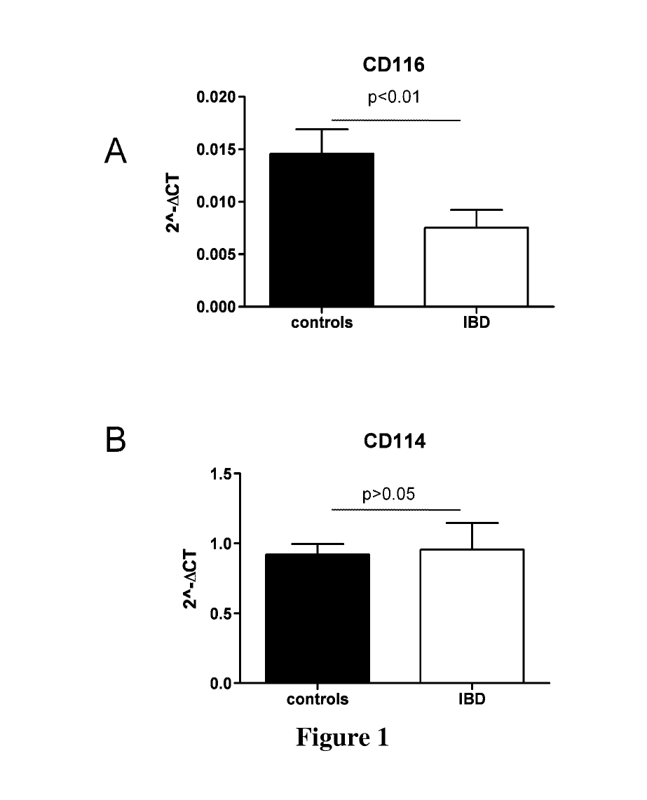

The present inventors have examined the expression levels of CD116 mRNA in IBD and control granulocytes. As shown in FIG. 1, real-time PCR analysis for CD116 and control CD114 (G-CSFR) were compared in ten healthy controls and ten patients with IBD. As can be seen, CD116 mRNA levels were decreased by nearly 60% in IBD granulocytes (p<0.001). CD114 levels were unchanged (p>0.05) between these two populations, demonstrating at least some degree of specificity for CD116.

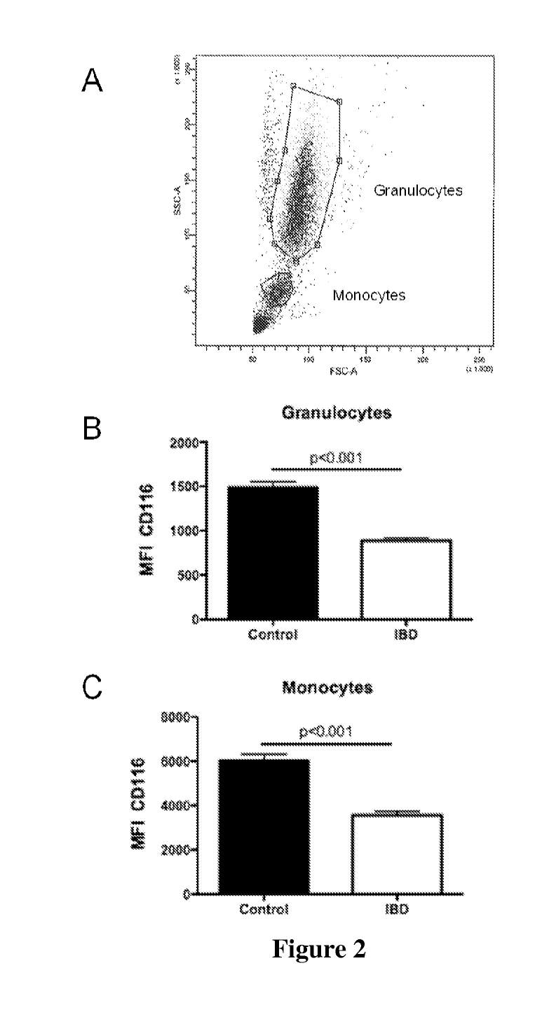

These findings were extended to define levels of surface CD116 on circulating granulocytes and monocytes in 52 IBD patients, 52 healthy controls and 8 disease control (IBS) patients by flow cytometry (see gating strategy in FIG. 2A). As shown in Table 1, the median age of IBD patients was 42 years old and 57% were male, for which 33 patients had Crohn's disease (CD) and 19 patients had ulcerative colitis (UC). The median age of normal patients was 36 years old and 54% were male. The median age of IBS patients was 46 years old and 17% were male.

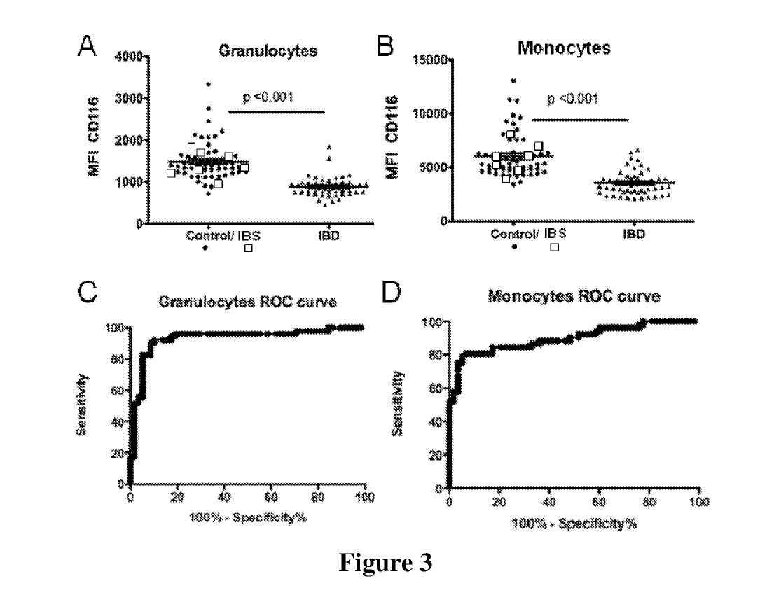

Flow cytometric analysis revealed that granulocyte surface CD116 levels in IBD (MFI 886±32) were significantly lower than healthy controls (MFI 1490±59, p<0.001, FIG. 2B). Likewise, monocyte CD116 levels in IBD (MFI 3566±156) were significantly less compared to healthy controls (MFI 6042±268, p<0.001, FIG. 2C). Circulating lymphocytes did not express detectable CD116. Expression of CD116 on disease control (IBS) granulocytes (FIG. 3A) and monocytes (FIG. 3B) were indistinguishable from healthy control leukocytes (p<0.001 for both compared to IBD leukocytes).

Analysis of these results using a receiver operating characteristic (ROC) curve revealed an area of 0.931 for granulocytes with a cutoff MFI less than 1120 translating to a sensitivity of 90% and a specificity of 91% for predicting IBD (FIG. 3C). The ROC curve for monocytes showed an area of 0.898 that with a cutoff of MFI less than 4250 translating to a sensitivity of 81% and a specificity of 94% for predicting IBD (FIG. 3D). Of the 52 IBD patients, 82% (43/52) had low granulocyte and low monocyte CD116 levels, 6% (3/52) had low granulocyte but normal monocyte CD116, and 6% (3/52) had normal granulocyte and low monocyte CD116 levels. Of the IBD patients, 3 patients (3/52) had normal granulocyte and normal monocyte CD116 levels. Of the healthy controls and IBS controls, 87% (45/52) had normal granulocyte and normal monocyte CD116 levels, 4% (2/52) had normal granulocyte and low monocyte CD116, and 9% (5/52) had low granulocyte and normal monocyte CD116 expression. Of the healthy controls and IBS controls, no patients had low granulocyte and low monocyte levels.

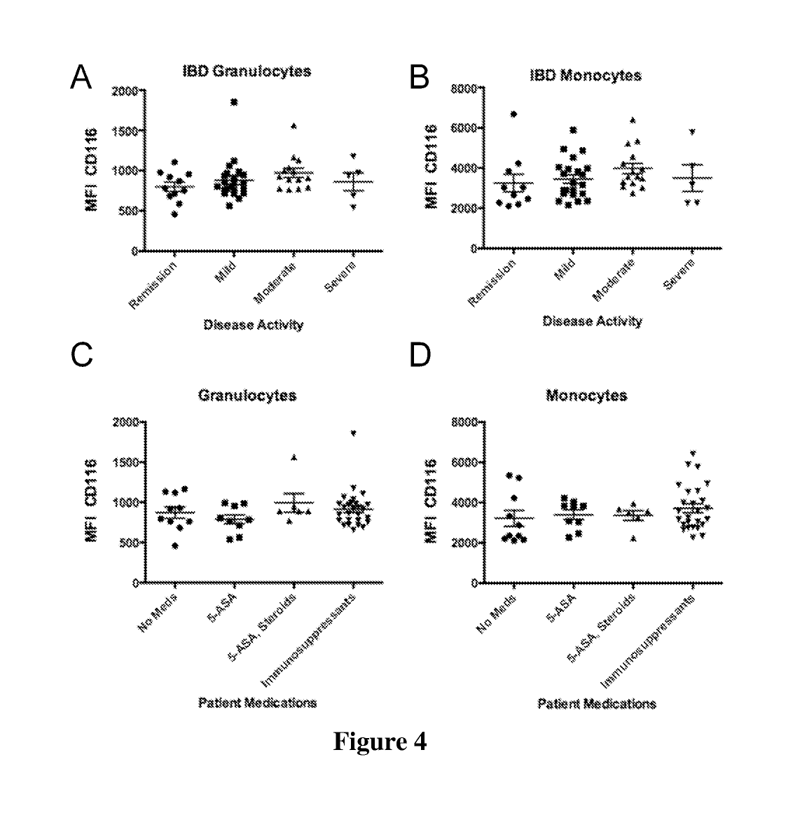

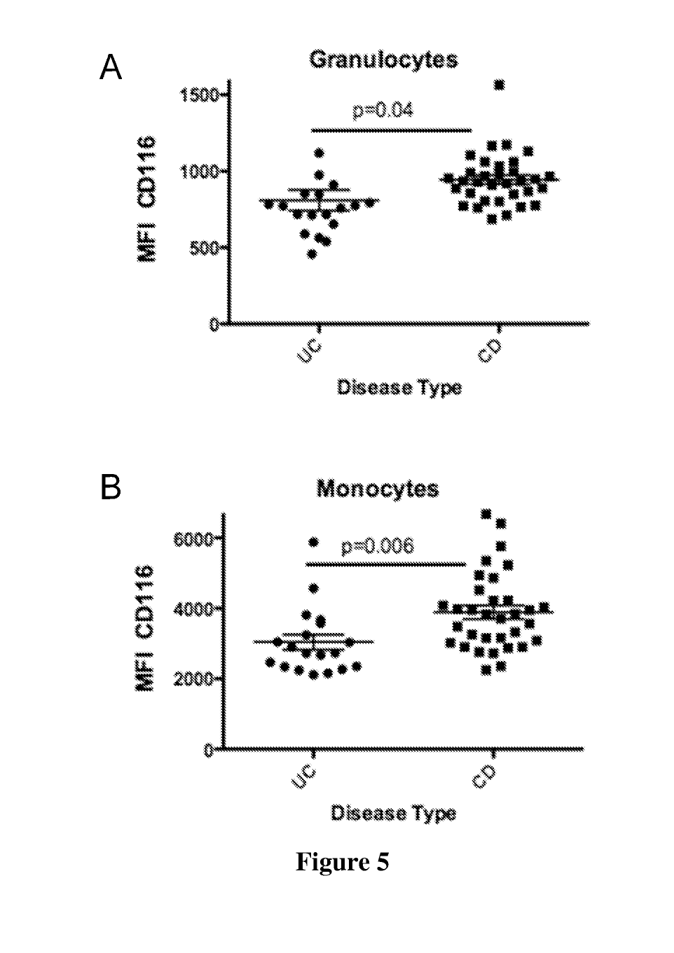

CD116 levels in IBD patients were not significantly affected by disease activity at the time of sampling (p>0.05, FIGS. 4A and 4B) and were independent of any medications (p>0.05, FIGS. 4C and 4D). Within IBD populations, UC patients were found to express significantly lower granulocyte (679±33 vs 897±35, p<0.001, FIG. 5A) and monocyte CD116 (2592±168 vs 3475±161, p<0.01, FIG. 5B) compared to CD patients.

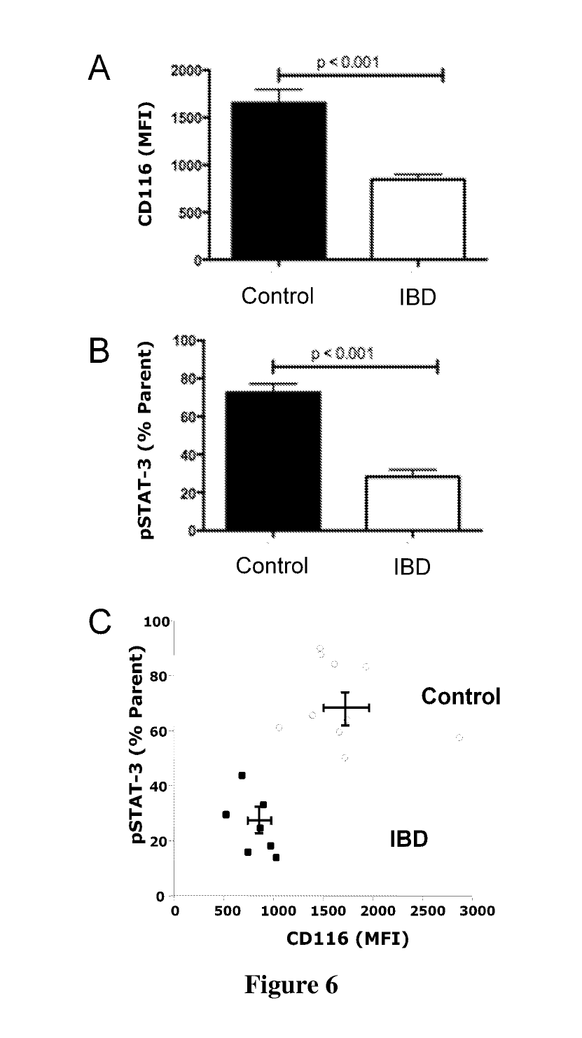

To determine if reduced levels of CD 116 correlated with a reduced functional response, GM-CSF activation (180 ng/ml for 15 min) of pSTAT3 was investigated in 10 healthy controls and 8 patients with IBD. In this analysis, it was reconfirmed that granulocyte surface CD116 levels in IBD (MFI 846±54) were significantly lower than healthy controls (MFI 1655±140, p<0.001) (FIG. 6A). Such decreased expression corresponded to significant reductions on STAT3 phosphorylation. Indeed pSTAT3 levels in IBD patients (28±4% positive) were significantly lower than healthy controls (73±5% positive, p<0.001) (FIG. 6B). A plot of these corresponding expression and function values showed a distinct separation between healthy controls and IBD (FIG. 6C). Discussion

The present inventors have discovered that decreased expression and function of CD116 is a distinguishing feature of IBD. Such a discovery provides important insight into the innate immune defects in IBD.

It is believed that Crohn's disease (CD) is associated with a significantly impaired acute inflammatory response and defects in bacterial clearance. Such defects result in granulomatous inflammation and persistence to the extent that some have suggested that CD is a primary immunodeficiency of unknown etiology. See, for example, Marks et al., Clin. Rev. Allergy Immunol., 2010, 38, 20-31. Central to the development and functional activation of innate immune cells is GM-CSF. As disclosed herein, the present inventors have discovered that CD116 expression is decreased on granulocytes and monocytes from both UC and CD patients. This repression was found to be generally independent of disease severity and current medications, suggesting that this defect is more basic in nature. Without being bound by any theory, it is believed that this phenotype is not likely to result from increased internalization or abnormal processing of CD116, since decreased surface expression paralleled decreases in CD116 mRNA expression.

Other aspects of the invention provide methods for treating IBD using recombinant GM-CSF. In some embodiments, analysis of CD116 can be performed prior to the initiation of rhGM-CSF therapy. Yet in other embodiments, expression of CD116 can be used as a stratifying tool for UC versus CD patients. As disclosed above, methods of the invention can distinguish a significant difference between CD and UC based on CD116 expression.

The present inventors have discovered functional abnormalities in IBD granulocytes administered exogenous GM-CSF, namely deficient GM-CSF-induced STAT3 phosphorylation. STAT3-mediated signaling in granulocytes has been shown to direct neutrophil migration, promote bacterial killing and to enhance neutrophil proliferation and survival. See, for example, Hamilton in Nat. Rev. Immunol., 2008, 8, 533-44. Without being bound by any theory, it is believed that this functional defect in STAT3 phosphorylation represents a direct reflection of decreased surface CD116 expression. As shown in FIG. 6, analysis plotting function (STAT3 phosphorylation) versus CD116 expression revealed a strong separation between the IBD and normal patient cohorts.

Decreased CD116 expression (e.g., in leukocyte) can be used as an IBD diagnostic marker as well as other clinical applications such as a therapeutic response predictor. It should be noted that conventional diagnosis of CD and UC is not straightforward and is often based on a combination of clinical, radiographic, endoscopic, and histological criteria. Patients often present with varying complaints of abdominal pain, rectal bleeding, weight loss, anemia and diarrhea that guide the diagnosis of IBD. Some of these symptoms overlap with other more common gastrointestinal disorders, including IBS and celiac disease. In cases where CD or UC is strongly suspected, early intervention could prove valuable. Methods of the invention can be used to distinguish between IBD and IBS. In the comparison of IBD to control, ROC curve analysis revealed sensitivity and specificity of nearly 90% for both monocytes and granulocytes in distinguishing IBD from healthy controls. These values compare well, and even exceed, the positive predictive value of currently available diagnostic tests. In addition, given the recent interest in sub-typing IBD patients prior to initiation of treatment, methods of the invention (e.g., the expression level of CD116) can also be used as a stratification tool for patient selection.

As shown herein, CD116 can be used as a biomarker for IBD. In addition, diminished CD116 expression and function indicate a role for GM-CSF-mediated signaling the pathogenesis of IBD as well as strategies for therapy and diagnosis.

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

1-22. (canceled)

23. A method for determining the presence of inflammatory bowel disease (IBD) in a subject comprising:
determining the expression level of granulocyte macrophage colony-stimulating factor receptor (CD 116) in a sample obtained from the subject; and
comparing the subject's CD116 expression level with a control CD116 expression level to determine the presence of IBD in the subject.

24. The method of claim 23, wherein the control CD116 expression level comprises a CD 116 expression level in a subject without IBD, and wherein a lower expression level of the subject's CD116 compared to the control CD116 expression level is an indication that the subject has IBD.

25. The method of claim 24, wherein the expression level of 50% or less of CD116 in the subject compared to the expression level of CD116 in the control subject is an indication that the subject has IBD.

26. The method of claim 23, wherein the control CD116 expression level comprises a CD116 expression level in a subject with IBD, and wherein a similar expression level of the subject's CD116 compared to the control CD116 expression level is an indication that the subject has IBD.

27. The method of claim 23, wherein the sample comprises blood, buccal swab, tissue, urine, or a combination thereof

28. The method of claim 23, wherein the sample comprises blood cells, granulocytes, monocytes, or a combination thereof

29. The method of claim 23, wherein the level of CD116 is determined using at least two antibodies capable of identifying CD116, contacting at least a portion of the sample obtained from the subject with the antibodies; and monitoring the extent of reaction between the contacted sample and the antibodies.

30. The method of claim 29, wherein the contacting and monitoring steps are carried out by extracting the proteins from the sample and conducting an assay to determine the quantity of CD116.

31. The method of claim 29, wherein the antigen-antibody binding reaction is detected by a technique comprising enzyme-linked immunosorbent assay (ELISA), radioimmunoassay (RIA), sandwich assay, Western blotting, immunoblotting analysis, an immunohistochemistry method, or a combination thereof.

32. The method of claim 29, wherein said contacting and monitoring steps are carried out by immunohistochemically reacting the sample and the antibodies and then detecting the reactions in the sample.

33. The method of claim 23 further comprising differentiating between ulcerative colitis (UC) and Crohn's disease (CD).

34. The method of claim 23, wherein the expression level of CD116 is detected using DNA microarray hybridization.

35. The method of claim 23, wherein the expression level of CD116 is detected using polymerase chain reaction (PCR).

36. A method for monitoring a patient's response to a treatment for an inflammatory bowel disease (IBD), said method comprising determining the expression level of granulocyte macrophage colony-stimulating factor receptor (CD116) in a sample taken from the patient, wherein increase in the expression level of CD116 of the patient compared to a reference expression level of CD 116 is indication that the patient is responding positively to the treatment.

37. The method of claim 36, wherein the reference expression level of CD116 comprises the pretreatment expression level of patient's CD116.

38. The method of claim 36 further comprising the steps of taking samples during the course of the treatment, and comparing the expression level of CD116 from the previously measured expression level of patient's CD116.

39. A method for determining a need for an invasive medical procedure to distinguish between inflammatory bowel disease (IBD) and irritable bowel syndrome (IBS) in a subject, said method comprising:
determining the expression level of the subject's granulocyte macrophage colony-stimulating factor receptor (CD116); and
determining a need for an invasive medical procedure by comparing the subject's CD116 expression level with the control CD 116 expression level.


SEQUENCE LISTING (6 sequences)

SEQ ID NO: 1 - DNA, 19 bp, Artificial Sequence
  CSF2R Forward PCR Primer
gcagacgtccgcatcttga

SEQ ID NO: 2 - DNA, 21 bp, Artificial Sequence
  CSF2R Reverse PCR Primer
ccgtcgtcagaaccaaattca

SEQ ID NO: 3 - DNA, 21 bp, Artificial Sequence
  CSF3R Forward PCR Primer
agccccaagtcctatgagaac

SEQ ID NO: 4 - DNA, 19 bp, Artificial Sequence
  CSF3R Reverse PCR Primer
gcaggagggggaagttgag

SEQ ID NO: 5 - DNA, 22 bp, Artificial Sequence
  Beta-Lactin Forward PCR Primer
gcactcttccagccttccttcc

SEQ ID NO: 6 - DNA, 22 bp, Artificial Sequence
  Beta-Lactin Reverse PCR Primer
caggtctttgcggatgtccacg